(19) United States
(12) Patent Application Publication
(10) Pub. No.: US 2022/0249978 A1
(43) Pub. Date: Aug. 11, 2022

(54) NOVEL SOLVENT FOR EXTRACTING, SOLUBILISING AND/OR FORMULATING VOLATILE AND NON-VOLATILE COMPOUNDS OF INTEREST IN ANIMAL NUTRITION AND HEALTH, PREPARATION METHOD AND USES THEREOF

(71) Applicants: ID4TECH, Valreas (FR); Avignon Université, Avignon (FR)

(72) Inventors: Francois GAUTIER, Seynod (FR); Camille ROZIER, Saint Maurice de Beynost (FR); Michel MAGNIN, Baud (FR); Farid CHEMAT, Moriéres les Avignon (FR); Anne-Sylvie FABIANO TIXIER, Rochefort du Gard (FR); Karine RUIZ, Les Valayans (FR)

(73) Assignees: ID4TECH, Valreas (FR); Avignon Université, Avignon (FR)

(21) Appl. No.: 17/438,095

(22) PCT Filed: Mar. 10, 2020
(86) PCT No.: PCT/EP2020/056297

§ 371 Date: Sep. 10, 2021

(30) Foreign Application Priority Data
Mar. 11, 2019 (FR) FR1902433

(51) Int. Cl. B01D 11/02; A61K 8/92

(52) U.S. Cl. CPC B01D 11/0288; A61K 8/922; B01D 11/0284

(57) ABSTRACT

A process of preparation of a solid and/or liquid totum or filtrate that includes placing in contact via mixing a vegetal matrix or part of a vegetal matrix with a solvent, extracting, solubilizing and/or formulating the mixture, optionally, filtering the mixture, and recovering the totum or the filtrate thus obtained. The solvent is glycerol monolaurate.

This invention concerns a process of preparation of a solid and/or liquid totum or filtrate from a biological matrix or part of a biological matrix and a solvent, as well as the totum or filtrate thus obtained according to said process, and its uses.

A totum can be defined as a mixture of a vegetal matrix preferably in powder form with a solvent. The totum thus includes active compounds or metabolites extracted or not from the vegetal matrix.

Active compounds or natural metabolites are molecules originating from a biological matrix or part of a biological matrix, usually a vegetal matrix, whose biological and technological activities have been demonstrated and described in the literature. These natural active compounds can be in pure form or contained in extracts. The interest of these active compounds can be established in the context of food and/or welfare and/or human or animal health. Their use as additives can cover a variety of purposes, such as:
improving health (anti-oxidant, anti-inflammatory, anti-microbial compounds, alkaloids and polyphenols),
improving palatability (compounds that increase palatability such as aromatic compounds, terpenes or pigments such as carotenoids or chlorophylls),
contribution to nutrition (nutrients such as proteins, amino acids, vitamins, trace elements, etc.).

The natural metabolites of vegetal origin to which it is possible to attribute biological activities of interest in food and human or animal health may belong to different families of molecules. These are mainly secondary metabolites, which, unlike primary metabolites, are not directly essential for plant nutrition, growth and development (Verpoorte, 2000, Secondary metabolism. In Metabolic engineering of plant secondary metabolism (pp. 1-29). Springer, Dordrecht). These are compounds whose biosynthetic pathways are fairly specific to a taxonomic group and which generally participate in the interaction mechanisms between the plant and its environment (defence, resistance and responses to abiotic and biotic stresses, symbioses, allelopathy, etc.).

There are different families of secondary metabolites of interest in animal nutrition and health.

The first is alkaloids (compounds that are generally alkaline and contain at least one nitrogen atom). These are compounds that generally have a significant biological activity, in particular an action on the central and/or peripheral nervous system (stimulant or depressant), notably as anaesthetics, as hypertensive agents or anti-hypertensive agents, as anti-malarial drugs or as anti-cancer drugs.

Alkaloids are generally grouped according to their nucleus (non-heterocyclic, indole derivative, pyrrole, pyridine, tropane, etc.). Alkaloids include well-known molecules such as caffeine, morphine, piperine, nicotine, atropine, scopolamine and quinine.

Capsaicinoids, including capsaicin and dihydrocapsaicin, can account for up to 90% of total capsaicinoids. These are the active components of the chilli pepper which belong to the benzylamine group of alkaloids. Consumption of capsaicin activates TRPV1 receptors which activate a burning sensation. It also stimulates the production of two hormones, adrenaline and noradrenaline, and therefore has therapeutic value given its anti-inflammatory, antioxidant and analgesic properties (Zimmer et al., 2012, Antioxidant and anti-inflammatory properties of Capsicum baccatum: from traditional use to scientific approach. Journal of Ethnopharmacology, 139(1), 228-233).

Then there are the carotenoid pigments (yellow, orange or red tetraterpenes), including carotenes, which are composed solely of carbon and hydrogen, and xanthophylls, which also contain oxygen atoms.

Chlorophylls (a, b, c1, c2 and d) are pigments present in all green plants (terrestrial and aquatic). Chlorophyll a (C₅₅H₇₂O₅MgN₄) is still the most common form found in plant leaves.

Anthocyanins are water-soluble pigments (oxygenated heterosides) that range from red to blue.

Curcuminoids (orange pigments from the rhizome of Curcuma longa), have been shown to significantly decrease concentrations of C-reactive protein, an important factor in inflammation (Sahebkar, Are Curcuminoids Effective C-Reactive Protein-Lowering Agents in Clinical Practice? Evidence from a Meta-Analysis. Phytother Res. 2013 Aug. 7. doi: 10.1002/ptr.5045).

Flavonoids can range in colour from red to ultraviolet depending on the pH and consist of two aromatic rings linked by three carbons.

These different classes of pigments have mainly inflammation-regulating and light-protecting effects and also act as antioxidants (powerful anti-free radicals) (Stahl and Sies, Bioactivity and protective effects of natural carotenoids. Biochimica et Biophysica Acta (BBA)-Molecular Basis of Disease, 1740(2), 101-107).

The interest of their use in animal feed is notably to couple their antioxidant activities with their participation in improving the visual quality (colouring and appearance) of the product formulation, as well as in the colouring and preservation of animal products (meat, eggs).

Terpenes are also interesting secondary metabolites. These are volatile compounds with an aromatic ring structure and hydroxyl and terpenoid groups. They are the source of the aromatic properties of certain plants according to their taxonomy. According to the literature, there are about 25,000 different terpene structures.

In addition to this, the properties of another family, the phenols, are essential components of essential oils.

Phenols are the metabolites that give essential oils their very characteristic smell and biological activities.

For example, oregano essential oil is composed mainly of thymol (phenol monoterpenoids) and its isomer, carvacrol and γ-terpinene, the presence of which gives the essential oil its antioxidant and antimicrobial properties.

Natural compounds of vegetal origin have a wide range of applications in the fields of cosmetics and perfumery, but also in health and human and animal nutrition. They are obtained by harvesting, drying, storing and packaging the raw vegetal material. Nevertheless, it is important to note that the extraction processes (solid/liquid) are at the heart of various controversies.

Essential oils can be obtained by hydrodistillation or steam distillation. Other processes are used to obtain more complex extracts than essential oils, such as cold maceration, hot digestion, decoction, leaching, percolation under pressure or cold, or infusion.

The most commonly used process is still the formation of oleoresin by extraction with volatile organic solvents such as petroleum ether, hexane, ethyl ether, acetone, carbon dioxide, benzene or toluene.

Although these solvents allow for a much more efficient and less selective extraction than water (extraction of mainly hydrophilic compounds) and are easily removed at the end of the process by simple evaporation, most of them are:
toxic, contributing to the contamination of soil and water,
flammable and
derived from non-renewable resources.

In addition, various studies have reported on the risks (carcinogenic and mutagenic in particular) incurred by prolonged exposure to these solvents.

In addition, the processes employed require a large amount of solvent and energy to achieve the desired yields.

Recently, European directives have been introduced to limit the use of this type of solvent with a view to protecting the environment (the fight against the greenhouse effect, soil contamination and the depletion of oil resources).

In the list of solvents to be limited, hexane is placed at the top of the scale. It is in fact a C6-saturated hydrocarbon that is marketed by distilling petroleum or natural gas. It belongs to the category of CMR type 3 molecules (carcinogenic, mutagenic and reprotoxic; EU CMR list). It is also highly flammable (flash point 23.3° C.), toxic to aquatic organisms, may impair fertility, causes skin irritation and may be fatal if swallowed or if it enters the respiratory tract.

In view of the above, there is now a need to develop or find new or alternative solvents to the controversial solvents listed above.

Some alternative solvents have already been proposed, and may be derived from different sources (wood, cereals, oilseeds). Terpene solvents are derived from the distillation of oleoresins (usually from pine) or by-products from other industries.

These include alcohols (C₆H₁₈O) and hydrocarbons (C₆H₁₀) which have many double bonds.

However, the use of these terpene derivatives is hindered by their chemical reactivity due to their double bonds. For example, limonene exposed to air undergoes auto-oxidation reactions which induce the formation of oxygenated hydroperoxide derivatives, increasing the allergenic character of this product and causing the formation of free radicals.

This is the case for bio-ethanol, which is obtained by fermenting sugars from the hydrolysis of field crops or co-products. Butanol and 1,3-propanediol are produced on the same principle. These are solvents commonly used in the pharmaceutical, cleaning and cosmetic industries.

Another agro-solvent is methyl-THF, a product of biosourced furfural (from maize and sugarcane residues, among other things).

It appears that the physico-chemical characteristics (lipophilic character, less volatile), the cost and the technological limitations of production of these alternative agro-solvents remain an obstacle to their use on an industrial scale.

In view of the above, one problem that this invention proposes to solve is to develop a new process of preparation of a solid and/or liquid totum or filtrate from a biological matrix or part of a biological matrix and a solvent that does not have the disadvantages listed above. In other words, by using a solvent that is notably non-toxic and environmentally friendly.

The first object of the solution to this problem is a process of preparation of a solid and/or liquid totum, which is a mixture of a vegetal matrix with a solvent, comprising:
a first step of contacting by mixing a vegetal matrix or part of a vegetal matrix with a solvent;
at least one second step which is an extraction, solubilisation and/or formulation phase of said mixture; and
a final stage of recovery of the totum thus obtained;
wherein the solvent is glycerol monolaurate.
The glycerol monolaurate should preferably be used at a temperature above 60° C., more preferably above 63° C.

The second object is a process of preparation of a solid and/or liquid filtrate comprising:
a first step of contacting by mixing a biological matrix or part of a biological matrix with a solvent;
at least one second step which is an extraction, solubilisation and/or formulation phase of said mixture;
a third step of filtering the mixture obtained in the second step; and
a final stage of recovery of the filtrate thus obtained;
   wherein the solvent is glycerol monolaurate.

The third object is the use of Glycerol Monolaurate taken alone or in mixture with one or more vegetable oils such as sweet almond oil, peanut oil, argan oil, avocado oil, calophyllum oil, safflower oil, rapeseed oil, coconut oil, wheat germ oil, jojoba oil, corn oil, hazelnut oil, apricot kernel oil, virgin olive oil, palm oil, grapeseed oil, castor oil, sesame oil, soybean oil, sunflower oil, oleic sunflower oil, non-oleic sunflower oil, and hydrogenated sunflower oil, as an extraction, solubilisation and/or formulation solvent.

The fourth object is a totum or filtrate that can be obtained by the process according to the invention.

The fifth object is the use of a totum or filtrate according to the invention, for the preparation of a food or cosmetic composition.

Finally, the last object is a composition comprising a totum or filtrate according to the invention, for its pharmaceutical use.

The invention relates to a process for the preparation of a solid and/or liquid totum or filtrate comprising glycerol monolaurate as a solvent.

Surprisingly, the Applicant was able to demonstrate that glycerol monolaurate, lauric acid monoglycerol, was a particularly advantageous alternative solvent with multiple interests. Glycerol monolaurate or 2,3-dihydroxypropyl decanoate has the following general formula:

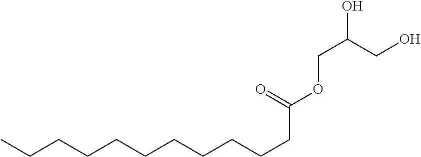

Glycerol monolaurate is a natural fatty acid monoester whose composition (more polar than some vegetable oils) allows the extraction of amphiphilic compounds such as capsaicinoids. Its use does not pose any risk to humans or animals. It is known that its melting point is above 60° C., more particularly 63° C. (CAS No. 142-18-7). It is therefore at this temperature that it is used as a solvent by the skilled person.

In addition, it shows pronounced antibacterial, antifungal and anti-inflammatory activities. It can be used as an antimicrobial agent and inhibits the growth of Candida strains in vitro and in vivo. It also acts against the growth of Gram+ but also Gram− bacteria such as Staphylococcus, Streptococcus, Gardnerella, Haemophilus but also Listeria monocytogenes. It also acts as a bacteriostatic agent, against Bacillus anthracis, i.e. it blocks its growth without killing the cells. In Staphylococcus aureus, glycerol monolaurate blocks the production of certain exoenzymes and virulence factors such as protein A, α-hemolysin, β-lactamase and toxic shock syndrome toxin 1 (TSST-1).

It would also reduce the risk of transmission and more specifically inhibit the signal transduction and inflammatory response due to HIV-1 and SIV infection.

Its action on the inflammatory cycle was also demonstrated as a parallel and significant decrease in the quantity of pro-inflammatory cytokines (IL-8 and TNF-α) was observed. Glycerol monolaurate can also act in synergy with other products, such as aminoglycosides, notably in the destruction of biofilm of antibiotic-resistant strains of Staphylococcus aureus. Indeed, pre-treatment with glycerol monolaurate would improve the response of biofilms to antibiotics.

The Applicant was able to identify glycerol as an interesting candidate for developing an extraction process for active compounds of natural origin.

For reasons of complexity of implementation, glycerol monolaurate has never been cited as a substitute for any extraction solvent in an associated process.

Indeed, although its physico-chemical properties make it an extraction solvent of choice, it is only for its antimicrobial and anti-inflammatory properties that it has been promoted until now.

Glycerol monolaurate is mentioned in international application WO2017181784 as a feed additive for laying hens.

International application WO2016169129 also describes the use of glycerol monolaurate in processes for the manufacture of antibacterial edible oil gel. Its antimicrobial properties also make it a component of choice in the treatment of viral, fungal and bacterial infections, as described in particular in international application WO2013159029.

In food applications, glycerol monolaurate is described in a process for treating raw fruit and vegetables. This is a 5 second to 30 minutes application of a solution of lactic acid and hydrogen peroxide or sodium benzoate or glycerol monolaurate. This process aims to reduce the presence of colonies of food-borne pathogens, such as certain known strains of Escherichia coli. An antimicrobial oil-in-water emulsion comprising glycerol monolaurate is also described in international application WO9531966. This emulsion has been developed for pharmaceutical purposes and shows an inhibitory action against a wide variety of infectious agents (bacteria, fungi, viruses).

As can be seen from the above paragraphs, it appears that glycerol monolaurate has been mentioned as a component in mixtures for food decontamination applications, infection treatment processes (in human health) or as a food additive. Its antimicrobial properties have been exploited in various fields of application. However, its use as a solvent in a complete process of extraction, solubilisation and/or formulation of vegetal material containing active natural metabolites of interest in animal supplementation has never been described. Its antimicrobial activity but also its physico-chemical properties enabling the extraction of a wide range of metabolites make it a candidate as a smart solvent, or solvagent, combining a notable efficiency as a solvent and providing the mixture with the antimicrobial and anti-inflammatory properties described above.

The invention and the advantages deriving therefrom will be better understood by reading the following description and the non-limiting methods of implementation, illustrated in relation to the annexed figures in which:

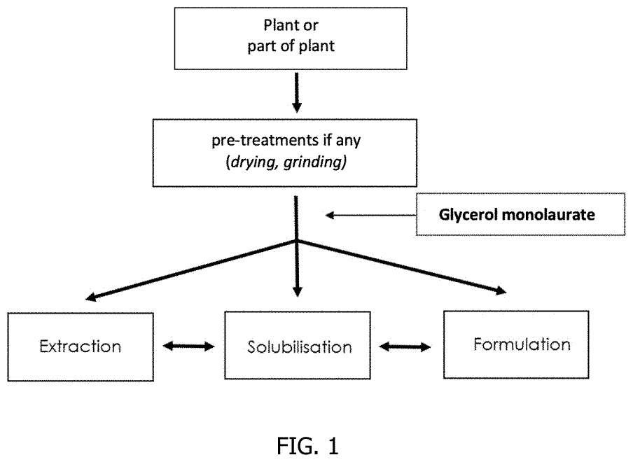

FIG. 1 shows a diagram of the process according to the invention using glycerol monolaurate (GML) as an innovative solvent in the extraction and/or solubilisation and/or formulation from a vegetal matrix which is a plant or part of a plant.

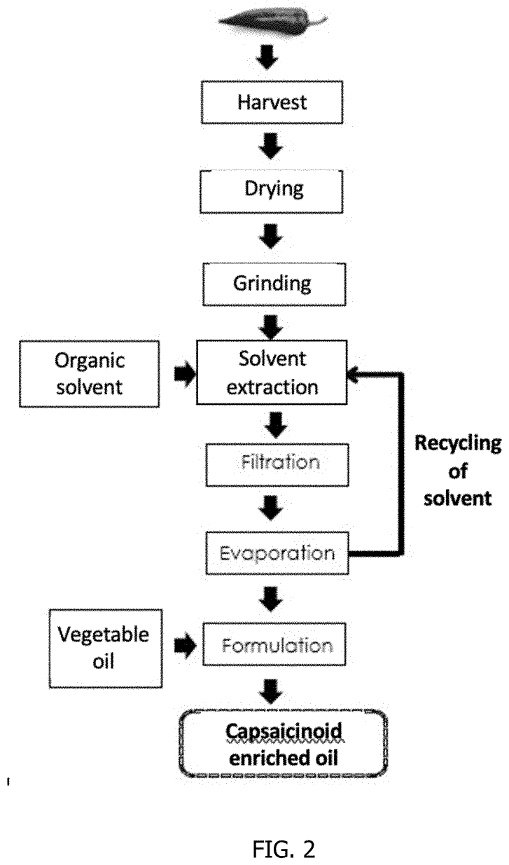

FIG. 2 is a diagram showing the steps for obtaining natural active compounds according to the known processes in the prior art.

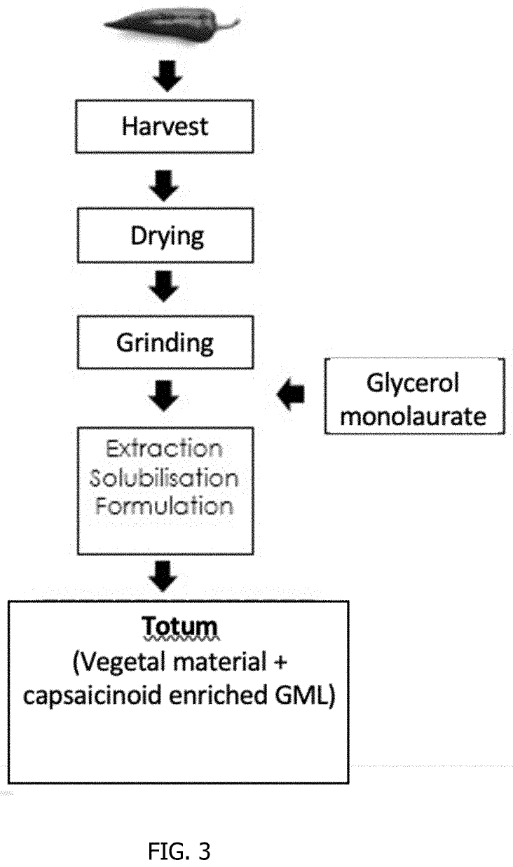

FIG. 3 is a diagram showing the steps for obtaining natural active compounds using glycerol monolaurate (GML) according to the process of the invention.

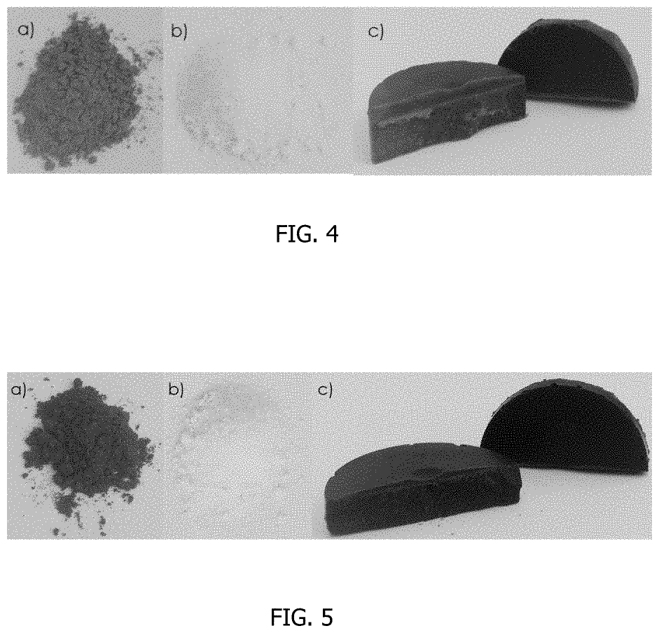

FIG. 4 is an image showing a) micronised chilli powder prior to the process; b) glycerol monolaurate; and c) chopped totum (chilli plant matrix and monolaurate glycerol loaded with metabolites of interest extracted from the chilli during the process) obtained by the process according to the invention.

FIG. 5 is an image showing a) micronised paprika powder prior to the process; (a), glycerol monolaurate (b), and c) the chopped totum (chilli plant matrix and glycerol monolaurate loaded with metabolites of interest extracted from the chilli during the process) obtained by the process according to the invention.

The invention relates to a process for preparing a solid and/or liquid totum or filtrate comprising a first step of contacting by mixing a biological matrix or part of a biological matrix with a solvent which is glycerol monolaurate as described above.

During the solubilisation and extraction steps of the process, the vegetal matrix, which may be in powder form, is brought into contact with the glycerol monolaurate and heated. The heating time and temperature are optimised according to the vegetal matrix. Preferably, the glycerol monolaurate is used at a temperature above 60° C., even more preferably above 63° C., even more preferably above 80° C. The totum is the sum/total of
1) glycerol monolaurate which has been loaded with active compounds extracted from the vegetal matrix (after extraction/solubilisation)
2) the vegetal matrix depleted in some of the active compounds it contained and which were transferred to the solvent (glycerol monolaurate) during extraction.

The totum forms a single mass containing in mixture the products 1) and 2) resulting from the said process. The therapeutic effect and biological activities of the total plant are superior to that of any of its constituents (coherent set of active principles with synergistic actions). It must be considered that the therapeutic effect and biological activities of the totum are even more significant since the active molecules of the vegetal matrix have been made more available by the extraction, and the biological activities of the intelligent solvent, glycerol monolaurate, are added to, or act in synergy with, those of the active ingredients extracted from the plant. It must also be considered that the vegetal matrix, in which certain molecules of interest in terms of activity have remained trapped as in any extraction, is preserved in the totum.

Filtrate is the term used for liquid material that has passed through a filter. As described above, this is 1) glycerol monolaurate loaded with active compounds extracted from the vegetal matrix. It is therefore the substance obtained after the totum has undergone a filtration step, allowing it to be freed from the powder formed from the biological matrix or part of the biological matrix.

The first step of the process consists of bringing the solvent, which is glycerol monolaurate, into contact, by mixing, with a biological matrix or part of a biological matrix.

The biological matrix or part of the biological matrix is preferably in powder form, i.e. in the form of a dry powder.

Preferably, the biological matrix or the part of the biological matrix is, taken alone or in a mixture, selected from alfalfa, artemisia, artichoke, ash (leaves), basil, bay, chamomile, chilli, cinnamon, clove, clover, coriander, cypress, dandelion, eucalyptus, fenugreek, garlic, goldenrod, juniper, lavender, lemon, lemon grass, nettle, orange, oregano, paprika, peppermint, pine, pepper, rosemary, sage, savory, tansy, thyme, turmeric, walnut (leaves), white mustard, wild thyme, wormwood, yarrow, or micro-organisms or media from cultures of micro-organisms.

Preferably, the biological material is a vegetal material. Vegetal material is a plant or part of a plant such as a leaf, fruit, stem, flower or root. As a non-limiting example of vegetal material or part of vegetal material that can be used, we can mention, taken alone or in a mixture, alfalfa, artemisia, artichoke, ash (leaves), basil, bay, chamomile, chilli, cinnamon, clove, clover, coriander, cypress, dandelion, eucalyptus, fenugreek, garlic, goldenrod, juniper, lavender, lemon, lemon grass, nettle, orange, oregano, paprika, peppermint, pine, pepper, rosemary, sage, savory, tansy, thyme, turmeric, walnut (leaves), white mustard, wild thyme, wormwood or yarrow.

Preferably, the biological matrix or part of a biological matrix is a vegetal material or part of a vegetal material selected from chilli, paprika, oregano and rosemary, taken alone or in a mixture.

As illustrated in FIG. 1, preferably the process according to the invention comprises a step, prior to the first step described above, of drying and/or grinding the biological matrix or part of a biological matrix, which is advantageously a vegetal matrix or part of a vegetal matrix as described above.

The process which is the subject matter of the invention comprises a second step which is an extraction, solubilisation and/or mixture formation phase between the biological matrix or part of the biological matrix and the solvent.

As shown in FIG. 1, the Applicant has demonstrated that the solvent glycerol monolaurate can be used to carry out three essential steps to obtain natural active compounds from a biological matrix or part of a biological matrix. These are the extraction, solubilisation and/or formulation phases. Glycerol monolaurate can be used to perform all three phases in series, only two of the three phases or a single phase independently of the others.

According to a particular embodiment of the invention, the second step of the process according to the invention comprises at least two of the three phases selected from the extraction, solubilisation and/or formulation phases of said mixture. Preferably, the two phases are extraction and solubilisation or extraction and formulation.

The extraction stage consists of separating elements (molecules) from a liquid (liquid/liquid extraction) or solid (solid/liquid extraction) matrix, usually using a solvent.

The classic extraction methods vary according to the desired composition of the extract: without a press-type solvent for oils or juices, with solvents selected according to their chemical properties (polarity, etc.), their cost, and their dangerousness. The classic example is extraction with hexane or ethanol with the intention of making oleoresins. The use of toxic or harmful solvents makes the evaporation step mandatory. According to the invention, eco-extraction with glycerol monolaurate allows the solvent, which is not harmful and has biological activities of interest, to be retained in the totum or filtrate resulting from the process. Extractions by supercritical CO₂ or by steam distillation (case of essential oils) are also commonly used. Extraction can be assisted by the use of ultrasound or microwaves.

The solubilisation step makes it possible to make the active compounds of interest, previously extracted and removed from the matrix, soluble in the extraction solvent, in other words, in this case, glycerol monolaurate. This is facilitated by heat, agitation and is highly dependent on the chemical properties of the extracted molecules and the solvent used.

Formulation consists of shaping the product resulting from the extraction and solubilisation stages and containing the active ingredients in a homogeneous manner, in order to target its field of use. It ultimately leads to the development of a product that can be marketed.

According to a preferred embodiment of the invention, the second step comprises the three phases of extraction, solubilisation and formulation of said mixture.

As can be seen from FIG. 3, the second step of the process according to the invention corresponds to the extraction, organic solubilisation and formulation steps of the usual additive production processes as shown in FIG. 2. With the process according to the invention, no co-products, no waste, from the vegetal material itself or from the solvent is generated.

One of the advantages of using this solvent in this process is the direct 3-phase-in-1 extraction/solubilisation/formulation procedure. As mentioned above, each phase (extraction, solubilisation or formulation) can be carried out independently of the other phases using glycerol monolaurate as the solvent.

Similarly, only two out of three of these steps can be carried out using said solvent. The invention relates in the first instance to the use of glycerol monolaurate as a solvent. In addition, the Applicant has, despite the difficulty of implementation, thought of the process associated with the use of said solvent. The major challenges overcome by the Applicant were to implement the process, to eliminate any degradation of the extracted compounds by glycerol monolaurate and to make the extraction/solubilisation and formulation efficient in terms of yield.

The process according to the invention optionally comprises a third step of filtering the mixture obtained in the second step. This filtration step allows the recovery, in the final step, of a final product free of biological or vegetable powder and therefore only a charged solvent which is a filtrate.

Alternatively, the process does not include this filtration step. The product thus recovered is called the totum.

The entire product generated by the process, either the totum or the filtrate, can be used in human food or animal feed, preferably in animal feed.

It should be noted that the basic materials used to carry out the said process are biological or vegetal material and the proposed novel solvent: glycerol monolaurate. Before the process is carried out, the active vegetal ingredients (metabolites known for their biological activities) are contained and retained by the biological or vegetal matrix (not or partially bioavailable). Said process including the steps of extraction and solubilisation of the vegetal actives allows the transfer of these metabolites from the vegetal matrix to the glycerol monolaurate. Extraction and transfer into glycerol monolaurate increases the bioavailability of these active ingredients and their absorption during transit in animals.

The different absorption sites can be modified by the formulation included in the process. Ultimately, the filtrate (glycerol monolaurate loaded with vegetal active ingredients) or the totum, corresponding to the remaining vegetal material (which may have retained a small part of its vegetal active ingredients, whether available or trapped in the matrix) and to the glycerol monolaurate highly loaded with active metabolites extracted from the vegetal matrix and rendered bioavailable, can be used.

The process which is the subject matter of the invention preferably comprises the following steps:
contacting the complex biological or vegetal matrix (dry material powder) with the solvent (100% solid glycerol monolaurate);
vacuum (about 700 mbar in order to avoid oxidation of the metabolites of interest) or not, stirring, heating of the vegetal material and the solvent (depending on the compounds of interest, which can range from about 80° C. to about 180° C.) for a period of time determined by the vegetal matrix (about 20 minutes to about 60 minutes);
recovery of the bulk totum, or filtration to use only the filtrate which is the charged solvent.

Advantageously, the process according to the invention is easy to implement because it is limited in the number of steps, which therefore limits the time constraints.

The process according to the invention also avoids complex logistics and the use of tools. The elimination of certain classic steps in the extraction of vegetal active ingredients also limits the handling of equipment and the cleaning of machines, and is once again part of an ecological and environmentally friendly approach. The equipment, solvents and materials needed to produce the final product are inexpensive and non-hazardous for the handler, and adaptable in terms of volume.

The invention also concerns the use of an intelligent solvent, known as a solvagent, glycerol monolaurate, in a unique 3-step-in-1 process for the extraction, solubilisation and formulation of natural active compounds from vegetal matrices.

Glycerol monolaurate can advantageously be mixed with vegetable oils without drastically altering the extraction yields but adding interesting properties to the totum obtained.

More particularly, the object of the invention is the use of Glycerol Monolaurate taken alone or in mixture with one or more vegetable oils such as sweet almond oil, peanut oil, argan oil, avocado oil, calophyllum oil, safflower oil, rapeseed oil, coconut oil, wheat germ oil, jojoba oil, corn oil, hazelnut oil, apricot kernel oil, virgin olive oil, palm oil, grapeseed oil, castor oil, sesame oil, soybean oil, sunflower oil, oleic sunflower oil, non-oleic sunflower oil, and hydrogenated sunflower oil, as an extraction, solubilisation and/or formulation solvent.

The Applicant was able to demonstrate that glycerol monolaurate can be used as a broad spectrum extraction solvent (polar, apolar, amphiphilic compounds).

The properties of the totum (solvent+vegetal matrix from which the active compounds have been extracted and solubilised) can be multiple: antimicrobial, anti-inflammatory, antioxidant, nutritional, addition of fragrance, flavour or colouring.

Glycerol monolaurate can impart emulsifying properties to the product resulting from this process (in particular the filtrate), which is of considerable interest in animal nutrition and supplementation, as it can increase growth performance and flesh quality in farm animals.

The filtrate (charged solvent) or the totum can be used as an additive in sectors such as food, human or animal health, as an active material and dynamized by the solvagent, which is glycerol monolaurate.

Said invention proposes the use of glycerol monolaurate as a new solvent which, in addition to being used for the extraction/solubilisation/formulation steps of a vegetal matrix, can be ingested by animals and humans and provide its own properties of interest to the final product.

Advantageously, the process based on the use of glycerol monolaurate is in line with current environmental protection and respect issues. The ultimate interest of these eco-extraction processes is to replace solvents with CMR risks such as hexane.

Among the active compounds of interest which are targeted, i.e. preferred, during the application of said process, a family of alkaloids should be mentioned: the capsaicinoids (capsaicins, dihydrocapsaicins, nordihydrocapsaicins).

By the process according to the invention, it is also possible to extract carotenoid pigments (capsanthin, capsorubin, zeaxanthin, β-carotene, β-cryptoxanthin, β-cryptoxanthin, antheraxanthin) and chlorophylls.

The process temperature may be adapted to extract and not to deteriorate aromatic and volatile molecules such as p-cymene, γ-terpinene, α-pinene, 1,8-cineole, cis-sabinene hydrate, linalool, camphor, borneol, terpinen-4-ol, trans-p-mentha-1(7),8-dien-2-ol, verbenone, bornylacetate, α-terpineol, carvone, thymol, carvacrol, piperitenone, eugenol, α-ylangene, carvacrol acetate, methyl-eugenol, caryophyllene, α-humulene, cis-calamenene, α-calacorene, caryophyllene oxide, 14-hydroxy-(Z) caryophyllene, abetatriene, 14-hydroxy-9-epi-(E) caryophyllene.

Moreover, all the extracted compounds can act in synergy with each other and with the solvagents (intelligent solvent) which is glycerol monolaurate, depending on their properties.

Given the physico-chemical properties of glycerol monolaurate, a wide range of molecules (polar, apolar, amphiphilic) can theoretically be extracted into the solvent and contribute to the biological activity of the totum. Examples of such compounds include, but are not limited to, fatty acids, vitamins, bones and amino acids.

The invention also relates to a totum or filtrate, that can obtained by the process according to the invention.

The final product, filtrate or totum, once it returns to room temperature, generally expands and hardens.

Due to the presence of glycerol monolaurate, but also of various pigments extracted during the process, the totum or filtrate has a smooth, coloured and shiny appearance.

If the extracted vegetal material comes from an aromatic plant (rosemary or oregano, to name but two), the totum may also have the characteristic smell of these plants. The Applicant has defined the contribution in terms of properties of glycerol monolaurate.

In this invention, the Applicant not only updates the use of a new intelligent solvent, called a solvagent, and its interest for applications in food and animal health but also demonstrates the interest of the 3-in-1 process of organic extraction/solubilisation/formulation.

The process is adaptable in terms of temperature, duration of contact between vegetal matrix and solvent, and volume. This makes it possible to extract a range of natural active compounds from a wide variety of vegetal species. Heat-sensitive compounds such as terpene-type aromatic compounds can thus be extracted and incorporated into the filtrate or totum. The proposed new solvent is also thermostable and retains its properties and non-toxicity during the process steps.

The invention also relates to the use of a totum or filtrate according to the invention, for the preparation of a food or cosmetic composition. Thus, the charged filtrate or totum can then be formulated according to the needs and target animals and offered for example for their different properties as feed additives for livestock. By way of non-limiting examples, the charged filtrate or totum is preferably in the form of a powder, granule, pebble, ointment, paste, capsule, microcapsule or tablet.

The invention's last object is a composition comprising a totum or filtrate according to the invention, for its pharmaceutical use.

### EXAMPLES

This invention will now be illustrated with the following examples.

### Example 1

### Implementation of a Method According to the Invention

The vegetal matrix in the form of dry material is ground beforehand.

About 20 g of vegetal material and about 20 g of glycerol monolaurate (1/1) are inserted into a flask, preferably a ground-neck flask.

The flask is installed on a rotary evaporator, and its contents are preferably protected from light.

A low rotation (less than 500 rpm, preferably less than 200 rpm) is then applied to the flask.

The pressure inside the balloon is preferably fixed:
from 700 mbar, for compounds sensitive to oxidation,
to 1000 mbar for aromatic compounds in particular.

The flask is then immersed in a heating bath at a temperature of between 80° C. and 180° C., depending in particular on:
the solubility of the active metabolites to be extracted;
the thermal sensitivity of these metabolites;
the time set to apply the process.

The heating and stirring time depends mainly on the vegetal material and the heating temperature. It can be set between 20 minutes and 60 minutes.

In order to increase efficiency in terms of yield, time and energy required, an optimum temperature and duration can be defined specifically for each vegetal material, depending in particular on the active metabolites targeted.

### Example 2

### Application of the Process According to the Invention on a Vegetable Matrix of Chilli (Capsicum) and Quantitative Analysis of the Capsaicinoids Transferred in the Filtrate Obtained

As shown in FIG. 4, examination of the resulting totum shows that the product resulting from the process according to the invention has a bright and smooth appearance, a deep red colour and a non-granular, heat-malleable texture.

This totum can be formulated quite easily depending on the target animal and the end use, especially when making a premix.

As described above, the family of alkaloids preferred for application of the process to a vegetal material such as chilli is the capsaicinoid family.

Analyses show that the micronised chilli powder used in said process contains 1211 mg/100 g of total capsaicinoids, divided into three main molecules: nordihydrocapsaicin, capsaicin, and dihydrocapsaicin.

As shown in Table 1 below, this equates to approximately 1.2% capsaicinoids in the vegetal matrix.

On average, 1017.3 mg/100 g of filtrate was extracted and transferred into the filtrate, i.e. 84% of the capsaicinoids in the vegetal matrix in terms of mass/mass ratio.

It should be noted that the residue analysed corresponds to the starting vegetal matrix taken en masse in a solidified glycerol monolaurate fraction, loaded with extracted compounds of interest that could not be filtered.

The interest of the process is to be able to keep:
the filtrate loaded with metabolites of interest; or
the totum containing the residual powder in which a portion of the active compounds of interest remain and said solvent having extracted the major part of the targeted active compounds and making them bioavailable upon ingestion.

The biological interest of capsaicinoids in animal supplementation is not negligible as they possess a range of properties recognised in the literature. As can be seen from the table below, capsaicin (8-methyl-N-vanillyl-6-nonenamide) accounts for about 45% of the total capsaicinoids, 43.6% in the present study.

In particular, it can be used commercially in the preparation of cosmetics or ‘hot’ ointments for its antimicrobial and pungent properties.

Chilli extracts are used as dietary supplements in specific diets and the anti-inflammatory activity of capsaicin is used in pharmaceutical creams.

In agronomy, resistance to certain fungal diseases appears to be correlated with capsaicinoid content.

When used on other crops, capsaicinoids have antimicrobial and antifungal effects, notably by inducing the stimulation of the synthesis of enzymes involved in plant defence such as chitinases.

TABLE 1
Capsaicinoid content (nordihydrocapsaicin, capsaicin, dihydrocapsaicin) of
micronised chilli powder, filtrates and filtration residues obtained by said process.

| | Means (mg/100 g of product) | | | |
|---|---|---|---|---|
| | Nordihydrocapsaicin | Capsaicin | Dihydrocapsaicin | Total |
| Chilli powder | 184 | 528 | 499 | 1211 |
| Filtrates (n = 3) | 156 ± 2.65 | 438.67 ± 10.21 | 422.33 ± 10.60 | 1017.33 ± 22.50 |
| Residue (n = 3) | 91.17 ± 3.53 | 259.33 ± 10.02 | 245.67 ± 7.77 | 599.33 ± 21.36 |

Results are expressed as mean±standard deviation (n=3). The residue corresponds to the starting vegetal matrix taken en masse in the solvent taken en masse, which could not be filtered. It therefore constitutes a vegetal powder fraction and a solvent fraction loaded with extracted compounds.

Chemical analyses for capsaicinoids were performed by UPLC (ACQUITY UPLC® (Waters)—C18 BEH column (2.1×50 mm)). UV detection was done specifically at 280 nm and coupling with a mass spectrometer allowed the identification and determination of capsaicin, dihydrocapsaicin, and nordihydrocapsaicin (by passing standards), respectively. Carotenoid determination was performed by UPLC and UV detection specifically at 470 nm. The coupling to mass spectrometry is carried out with the spectrometer described above equipped with an APCI (Atmospheric Pressure Chemical Ionisation) source. The aromatic compounds were determined by GC-MS (Agilent 7890A and 7000A, Santa Clara, USA) with a 60-metre DB-5 MS column.

### Example 3

### COSMO-RS Simulation and Physical Properties of the Solvent

Solubilisation studies of different compounds were carried out by simulation using the COSMO-RS software to predict the solubilisation of active substances in hexane or in glycerol monolaurate.

It appears that glycerol monolaurate extracts the various compounds better when heated to temperatures above 100° C.

For the study of aromatic compounds, a study at 80° C. was carried out in order to try to limit the degradation of the latter.

As can be seen from Table 2 below, it can be observed that overall glycerol monolaurate extracts as well or better than hexane.

### Example 4

### Application of this Process to a Paprika Vegetal Matrix (Capsicum) and Quantitative Analysis of Carotenoids Transferred to the Filtrate

As can be seen from FIG. 5, examination of the resulting totum shows that the product resulting from the process has a dark orange colour, a shiny and smooth appearance and a non-granular, heat-malleable texture.

Like the totum obtained when applying the process with chilli, the paprika totum can be transformed quite easily depending on the target animal and the demand when making a premix.

Table 3 below details the total free carotenoid content after saponification of the samples (in μg/g). Results are expressed as mean±standard deviation (n=3).

TABLE 3:

| | | Paprika | Paprika |
|---|---|---|---|
| | | residue/GML | filtrate/GML |
| | Paprika powder | (n = 3) | (n = 3) |
| Capsanthin | 456.6 ± 87.8 | 156.7 ± 43 | 233.2 ± 43.3 |
| Capsorubin | 91.7 ± 23.6 | 15.8 ± 4.2 | 8.2 ± 2.1 |
| Zeaxanthin | 79.6 ± 13.1 | 37.5 ± 4.0 | 75.2 ± 12.3 |
| β-Carotene | 69.6 ± 6.7 | 37.5 ± 5.8 | 85.2 ± 12.4 |
| β-Cryptoxanthin | 27.4 ± 6.1 | 16 ± 1.2 | 34.6 ± 4.9 |
| Antheraxanthin | 96.8 ± 15.8 | 60.9 ± 19.8 | 85.5 ± 16.6 |
| Total carotenoids | 821.8 ± 139.0 | 324.5 ± 73.7 | 522 ± 91.1 |

The carotenoid profile of paprika extracts shows the presence of carotenoid esters in the form of monoesters and diesters.

A small amount of carotenoids in free form is also identified in these extracts.

Saponification is carried out in order to quantify the carotenoids. The clearly preponderant carotenoid in paprika is capsanthin, which accounts for almost 56% of the total carotenoids identified in paprika powder.

Other carotenoids identified were antheraxanthin (11.8%), capsorubin (11.1%), zeaxanthin (9.7%), β-cryptoxanthin (3.3%), and β-carotene (8.5%).

In the filtrate obtained by said process, 63.5% of the total carotenoids were extracted and transferred and 51% concerning the major carotenoid, capsanthin.

As in the example above for chilli, it should be noted that the residue analysed corresponds to the starting vegetal matrix taken en masse in a solidified glycerol monolaurate fraction, loaded with extracted compounds of interest that could not be filtered.

Thus, it appears that the conservation of the totum at the end of the process can be of considerable interest.

Carotenoids can be used as a colouring agent for meat and by-products (eggs) in animal nutrition, which is of particular benefit to consumers. In addition, carotenoids are powerful antioxidants that have the ability to absorb aggressive blue radiation and prevent the production of ROS (Reactive Oxygen Species) and thus associated cell damage.

This protective activity of vegetal cells is linked to their structure (double bonds, keto group and sometimes 5-centre ring).

The presence of these compounds is therefore advantageously targeted in said process.

### Example 5

### Application of this Process on an Oregano Vegetal Matrix and Qualitative Analysis of the Aromatic Compounds Transferred in the Filtrate

The process described in Example 1 is applied to an oregano vegetal matrix.

Of the total number of extracted oregano metabolites identified in the hexane extract (=100%), 63.5% of the metabolites were also identified in the oregano essential oil. There is therefore a 63.5% recovery in qualitative terms between the essential oil of oregano and the extract of oregano with a hexane type solvent.

Of the totality of metabolites extracted from oregano and identified in the hexane extract (=100%), 34.1% of the compounds present were also identified in the filtrate obtained during said process. 34.1% of the metabolites extracted in a hexane extract (transferred from matrix to hexane) were transferred from the oregano vegetal matrix to glycerol monolaurate. These include metabolites with biological activity of interest. The remaining metabolites are retained in the ‘depleted’ powder, which is itself present in the totum.

As shown in Table 4 below, these include p-cymene, y-terpinene, cis-sabinene hydrate, linalool, borneol, terpinen-4-ol, α-terpineol, carvone, thymol, carvacrol, eugenol, carvacrol acetate, caryophyllene, α-humulene, caryophyllene oxide, 14-hydroxy-(Z)-caryophyllene and abetatriene.

Carvacrol is the major compound in the essential oil of this rosemary species and is the most transferred compound in the filtrate.

Together with thymol, it is one of the compounds that plays a major role in the biological antioxidant and antimicrobial activities of oregano essential oil.

Other compounds, including glycerol monolaurate, were specifically annotated in the filtrate. They participate in the biological activity of the totum and make the interest of this process in the dynamisation of the totum.

For the compounds that were not annotated in the filtrate and were annotated in the hexane extract, two possibilities can be considered:
either they are not transferred to the filtrate
or that they are, but in too small quantities, compounds that would therefore be below the detection limit of the mass spectrometer.

Table 4 below shows the qualitative aspect of the transfer of aromatic compounds from an oregano vegetal matrix into the filtrate of the process (n=3) in comparison with a hexane extract of the rosemary vegetal matrix and an essential oil of the same vegetal material.

In table 4, the compounds marked fhe, he, h and f respectively are non-annotated compounds using IR and ADAMS, found in the filtrate, essential oil and hexane extract (fhe1-11), in the essential oil and hexane extract of rosemary (he1-4), specifically in the hexane extract (h1-25) and specifically in the filtrate (f1-33). The “x” represents the qualitative presence of the compound in the extract and the “−” represents its absence.

TABLE 4

| | | | Extract |
|---|---|---|---|
| | | | according to |
| | Rosemary | Rosemary | process of |
| Molecules identified | hexane extract | essential oil | invention |
| α-pinene | x | x | — |
| camphene | x | x | — |
| octen-3-ol | x | x | — |
| beta pinene | x | x | — |
| 3-octanol | x | x | — |
| α-phellandrene | x | x | — |
| δ-3-carene | x | x | — |
| α terpinene | x | x | — |
| p-cymene | x | x | x |
| limonene | x | x | — |
| (Z)-6-ocimene | x | x | — |
| y-terpinene | x | x | x |
| cis-sabinene hydrate | x | x | x |
| terpinolene | x | x | — |
| cymenene | x | x | — |
| linalool | x | x | x |
| 1,8 cineole | x | — | — |
| phenyl ethyl alcohol | x | — | — |
| cis-p-menth-2-en-1-ol | x | x | — |
| borneol | x | x | x |
| terpinen-4-ol | x | x | x |
| p-cymen-8-ol | x | — | — |
| α-terpineol | x | x | x |
| cis-sabinene hydrate acetate | x | — | — |
| neral | x | x | — |
| carvone | x | x | x |
| carvone oxide | x | — | — |
| geraniol | x | x | — |
| thymol | x | x | x |
| carvacrol | x | x | x |
| thymol acetate | x | x | — |
| eugenol | x | x | x |
| carvacrol acetate | x | x | x |
| caryophyllene | x | x | x |
| α-humulene | x | x | x |
| y-muurolene | x | — | — |
| y-cadinene | x | — | — |
| 6-bisabolen | x | x | — |
| spathulenol | x | x | — |
| caryophyllene oxide | x | x | x |
| humulene epoxide | x | x | — |
| caryophylla-4(12),8(13)-dien- | x | x | — |
| 5alpha-ol | | | |
| 14-hydroxy-(Z) caryophyllene | x | x | x |
| kaurene | x | x | — |
| abietatriene | x | x | x |
| fhe1 to 11 | x | x | x |
| he1 to 4 | x | x | — |
| h1 to h25 | x | — | — |
| f1 to f33 (including glycerol | — | — | x |
| monolaurate) | | | |

### Example 8

### Application of this Process on a Rosemary Vegetal Matrix and Qualitative Analysis of the Aromatic Compounds Transferred in the Filtrate

The examination of the filtrate obtained is conclusive as the filtrate has retained the specific aromas of rosemary (olfactory examination). It is also smooth and shiny and the green colour is characteristic of the rosemary vegetal matrix. It is strong enough to be formulated afterwards (mix) but also fatty enough to be easily ingested by livestock.

Regarding the application of said process on rosemary, the study carried out on the aromatic compounds transferred in the filtrate was done in a qualitative way (the measurement corresponds to the number of molecules identified and not to their quantity). Of the totality of metabolites extracted from rosemary and identified in the hexane extract (=100%), 25% of the compounds present were also identified in the filtrate obtained during said process. These are the compounds that are predominantly present (in terms of concentration) in the hexane extract and in the vegetal matrix and that are responsible for the plant's biological activity. The remaining metabolites are retained in the ‘depleted’ powder, which is itself present in the totum.

As shown in Table 5 below, it was advantageous to find in the filtrate: α-pinene, 1,8-cineole, linalool, camphor, borneol, terpinen-4-ol, α-terpineol, trans-p-mentha-1(7),8-dien-2-ol, verbenone, bornyl acetate, piperitenone, eugenol, α-caryophyllene, α-humulene, cis-calamenene, α-calacorene, 14-hydroxy-9-epi-(E)-caryophyllene, hexadecanoic acid.

For compounds that have not been annotated, two possibilities are to be considered, as for oregano:
either they are not transferred to the filtrate
or in too small quantities in the filtrate, which would therefore be below the detection limit of the mass spectrometer.

Table 5 below shows the qualitative aspect of the transfer of aromatic compounds from a rosemary vegetal matrix into the filtrate of the process (n=3) in comparison with a hexane extract of the rosemary vegetal matrix.

The compounds marked fh, h, and f respectively are non-annotated compounds using IR and ADAMS, found in the filtrate and hexane extract (fh1-16), specifically in the hexane extract (h1-75) and specifically in the filtrate (f1-29). The “x” represents the qualitative presence of the compound in the extract in question and the “−” represents its absence.

TABLE 5

| | Rosemary | Extract according |
|---|---|---|
| | hexane | to process of |
| Molecules identified | extract | invention |
| α-pinene | x | x |
| camphene | x | — |
| sabinene | x | — |
| 6-pinene | x | — |
| myrcene | x | — |
| α-terpinene | x | — |
| p-cymene | x | — |
| limonene | x | — |
| 1,8-cineole | x | x |
| y-terpinene | x | — |
| terpinolene | x | — |
| p-cymenene | x | — |
| linalool | x | x |
| 2,6-dimethylphenol | x | — |
| chrysanthenone | x | — |
| α-campholenal | x | — |
| camphre | x | x |
| camphene hydrate | x | — |
| δ-terpineol | x | — |
| borneol | x | x |
| terpinen-4-ol | x | x |
| 3-carene | x | — |
| α-terpineol | x | x |
| trans-p-mentha-1(7),8-dien-2-ol | x | x |
| verbenone | x | x |
| trans-carveol | x | — |
| carvone | x | — |
| linalyl acetate | x | — |
| bornyl acetate | x | x |
| thymol | x | — |
| carvacrol | x | — |
| piperitenone | x | x |
| eugenol | x | x |
| linalool isobutanoate | x | — |
| α-ylangene | x | x |
| methyl-eugenol | x | x |
| 6-isocomene | x | — |
| α-caryophyllene | x | x |
| α-humulene | x | x |
| α-amorphene | x | — |
| δ-amorphene | x | — |
| y-cadinene | x | — |
| δ-cadinene | x | — |
| cis-calamenene | x | x |
| α-calacorene | x | x |
| δ-alacorene | x | — |
| Z-isoeugenol acetate | x | — |
| 14-hydroxy-9-epi-(E)-caryophyllene | x | x |
| cadalene | x | — |
| 14-hydroxy-α-humulene | x | — |
| acide hexadecanoique | x | x |
| fh1 to fh16 | x | x |
| h1 to h75 | x | x |
| f1 to f29 (including glycerol monolaurate) | — | x |

### Example 9

### Use of Glycerol Monolaurate in a Mixture with a Vegetable Oil and Application of this Process for the Extraction, Solubilisation and Formulation of a Paprika Powder

The efficiency in terms of β-carotene yield of mixtures of glycerol monolaurate and hydrogenated sunflower oil was tested by applying the diagram of said process. An experimental design involving different temperatures applied to the process and different ratios of glycerol monolaurate to hydrogenated sunflower oil was used to evaluate the criteria determining the efficiency of the process.

As shown in Table 6, temperature is indeed a parameter that significantly modulates (p<0.005, ANOVA) the extraction yield of β-carotene, especially from paprika powder (Table 6).

Conversely, modulating the percentage of glycerol monolaurate mixed with hydrogenated sunflower oil does not significantly modify the extraction yield of the process (p>0.1, ANOVA).

It is therefore possible to use glycerol monolaurate, the new extraction/solubilisation/formulation solvent proposed by the Applicant, in mixture with a vegetable oil which can likewise contribute its biological properties to the filtrate or totum obtained by said process.

Table 5 below is an ANOVA of the experimental design of said process set up to evaluate the possibility of using glycerol monolaurate (GML) in mixture with a vegetable oil (hydrogenated sunflower oil) for the extraction/solubilisation/formulation of paprika.

TABLE 6

| | Responses | | | | | |
|---|---|---|---|---|---|---|
| | mg β-carotene/ | | mg β-carotene/ | | mg β-carotene/ | |
| | 100 g totum | | 100 g MS | | 100 g filtrate | |
| Variables | F-ratio | p-value | F-ratio | p-value | F-ratio | p-value |
| Temperature | 14.10 | 0.0038 | 75.26 | 0.0000 | 31.07 | 0.0002 |
| % GML | 0.59 | 0.4604 | 0.58 | 0.4652 | 0.33 | 0.5763 |

### Example 10

### Use of Glycerol Monolaurate and its Application to Microwave Technology

A microwave-assisted heating and extraction experiment demonstrated that glycerol monolaurate was rendered liquid by microwave heating and that the compounds in the vegetal raw material, whether chilli, paprika, oregano or rosemary, could be extracted/solubilised and formulated using this eco-extraction technique and by integrating it into the process.

The Applicant has observed that this technology saves time and energy for a similar quality of products obtained.

### Example 11

### Use of Glycerol Monolaurate and its Application to Ultrasound Technology

Similarly, an ultrasound assisted extraction experiment demonstrated that said process using glycerol monolaurate could incorporate ultrasound assisted extraction technology for the extraction/solubilisation/formulation of a vegetable matrix of chilli, paprika, oregano or rosemary.

The integration of this eco-extraction technology reduces the time required for this process and thus reduces the energy costs of this process.

1. A process of preparation of a solid and/or liquid totum which is a mixture of a vegetal matrix with a solvent, comprising:
contacting by mixing a vegetal matrix or part of a vegetal matrix with a solvent to form a mixture;
extracting, solubilizing, and/or formulating said mixture; and
recovering the totum thus obtained;
wherein the solvent is glycerol monolaurate.

2. (canceled)

3. The process according to claim 1, comprising drying and/or grinding the vegetal matrix or a part of the vegetal matrix.

4. The process according to claim 1, wherein the vegetal matrix, part of the vegetal matrix is, taken alone or in a mixture, selected from alfalfa, artemisia, artichoke, ash (leaves), basil, bay, chamomile, chilli, cinnamon, clove, clover, coriander, cypress, dandelion, eucalyptus, fenugreek, garlic, goldenrod, juniper, lavender, lemon, lemon grass, nettle, orange, oregano, paprika, peppermint, pine, pepper, rosemary, sage, savory, tansy, thyme, tumeric, walnut (leaves), white mustard, wild thyme, wormwood, or yarrow.

5. The process according to claim 1, the second step comprises comprising at least two of extracting, solubilizing, and formulating said mixture.

6. The process according to claim 4, extracting, solubilizing, and formulating said mixture.

7. (canceled)

8. A totum obtained by the process of claim 1.

9. (canceled)

10. A pharmaceutical composition comprising a totum according to claim 8.

11. The process of claim 1, wherein the vegetal matrix comprises chilli, paprika, oregano or rosemary.

12. The totum of claim 8, wherein the totum is in powder, granule, pebble, ointment, paste, capsule, microcapsule, or tablet form.

13. A process of preparation of a solid and/or liquid filtrate comprising:
filtering the totum of claim 8; and
recovering the filtrate thus obtained.

14. The process of claim 1, wherein the glycerol monolaurate is in a mixture with a vegetable oil selected from the group consisting of sweet almond oil, peanut oil, argan oil, avocado oil, calophyllum oil, safflower oil, rapeseed oil, coconut oil, wheat germ oil, jojoba oil, corn oil, hazelnut oil, apricot kernel oil, virgin olive oil, palm oil, grapeseed oil, castor oil, sesame oil, soybean oil, sunflower oil, and combinations thereof.

15. The process of claim 14, wherein the vegetable oil is sunflower oil, and the sunflower oil is an oleic sunflower oil, non-oleic sunflower oil, or a hydrogenated sunflower oil.

16. A food comprising a totum according to claim 8.

17. The food of claim 16, wherein the food is human food or animal feed.

18. A cosmetic composition comprising a totum according to claim 8.

19. The cosmetic composition of claim 18, wherein the composition is an ointment.
